(19) United States
(12) Patent Application Publication
(10) Pub. No.: US 2015/0057354 A1
(43) Pub. Date: Feb. 26, 2015

(54) METHOD OF MAINTAINING OR IMPROVING TACTICAL PERFORMANCE AND COGNITIVE FUNCTION THROUGH DIETARY SUPPLEMENTATION

(71) Applicant: Natural Alternatives International, Inc., San Marcos, CA (US)

(72) Inventor: Jay R. Hoffman, Oviedo, FL (US)

(21) Appl. No.: 14/464,445

(22) Filed: Aug. 20, 2014

Related U.S. Application Data
(60) Provisional application No. 61/867,997, filed on Aug. 20, 2013.
(60) Provisional application No. 61/879,943, filed on Sep. 19, 2013.

(51) Int. Cl. A61K 31/197; A23L 1/29

(52) U.S. Cl. CPC A61K 31/197; A23L 1/293; A23V 2002/00
USPC 514/561; 426/2

(57) ABSTRACT

The present invention provides methods of maintaining or improving tactical performance and/or psychomotor performance, wherein the free amino acid beta-alanine, or a salt or ester thereof, is administered to an individual as a human dietary supplement over a period of time in an amount effective to maintain or improve tactical performance and/or psychomotor performance under normally stressful and/or fatiguing high intensity physical activity.

### FIELD OF THE INVENTION

The present invention relates to dietary supplementation and physiology, and, more specifically, to methods of maintaining or improving physiological responses to physical stress and maintaining or improving tactical performance and/or psychomotor performance affected by stress or fatigue.

### BACKGROUND OF THE INVENTION

Investigations examining β-alanine (herein also referred to as beta-alanine and BA) ingestion have been consistent in demonstrating significantly enhanced athletic performance during high intense activity (e.g., resistance exercise, repeated sprints) to a greater magnitude than a placebo (Hill et al., 2007; Hoffman et al., 2006; 2008a; 2008b; 2012; Kendrick et al., 2008; Stout et al., 2006; 2007). The efficacy of β-alanine ingestion appears centered on its ability to enhance the quality of a workout and sport performance by delaying skeletal muscle fatigue when supplemented in an effective amount over a sufficient period of time as with a dietary supplement. The ergogenic properties of β-alanine by itself appear to be very limited, but when consumed in sufficient dosages over time, β-alanine combines in the skeletal muscle with L-histidine to form the dipeptide carnosine (beta-alanylhistidine) and appear to have ergogenic effects (Dunnett and Harris, 1999). The primary role of carnosine is in the maintenance of acid-base homeostasis through enhanced intra-muscular hydrogen ion (H⁺) buffering capacity (Harris et al., 2006). Increasing intra-muscular carnosine concentration through β-alanine supplementation has demonstrated ergogenic potential for maximal exercise lasting 60 sec-240 sec (Hobson et al., 2012). Because carnosine is located in other tissues in addition to skeletal muscle, such as the brain and heart, it may also have additional physiological roles.

Previous research has shown that intense military training of durations from one to eight weeks can result in significant decreases in strength and power (Nindl et al., 2007; Welsh et al., 2008). In addition to the fatiguing effects associated with intense military training, decreases in shooting performance (Evans et al., 2003) and cognitive function (Lieberman et al., 2005) have also been reported. To defend against the physical and cognitive performance decrements related to intense and sustained military action, several studies have examined the efficacy of various stimulants and other pharmacological agents (Estrada et al., 2012; Gillingham et al., 2004; Lieberman et al., 2002). These studies have shown that such intervention can be very effective in sustaining military performance. Concerns have been raised regarding the safety and potential side effects associated with pharmaceutical agents, and calls for a greater effort in exploring non-pharmacological alternatives for military populations have been published (Russo et al., 2008). Despite the popularity of nutritional supplements in both deployed and garrisoned soldiers (Cassler et al., 2013; Lieberman et al., 2010), little is known regarding the efficacy of many of these supplements as they relate to specific military performance.

Several studies have suggested that carnosine may serve as a neuroprotector (Boldyrev et al., 2010; Stout et al., 2008). Carnosine's biological role as an antioxidant, an antiglycating and ion-chelating agent suggests that it may have a potential role in neuroprotection during oxidative stress. In addition, recent research has demonstrated that β-alanine can increase carnosine concentrations in the brain resulting in a decrease in serotonin concentrations, increase brain-derived neurotrophic factor (involved in the growth and differentiation of new neurons and synapses), and provide possible antianxiety-like effects (Murakami and Furuse, 2010). Thus, increases in carnosine concentration in the brain may also provide a benefit in maintaining focus, alertness and cognitive function during and after highly fatiguing, high intense activity.

The following art is incorporated by reference in its entirety, but especially for the information characterized by the title, which would be easily understood by one of skill in the art. Artioli G. G., Gualano B., Smith A., Stout J. R., Junior A. H. L., The Role of β-alanine Supplementation on Muscle Carnosine and Exercise Performance Med Sci Sports Exerc 42(6):1162-1173, 2010; Baguet, A., et al. (2010). “Important role of muscle carnosine in rowing performance.” Journal of applied physiology 109(4): 1096-1101; Boldyrev A. A., Stvolinsky S. L., Fedorova T. N., Suslina Z. A., (2010) Carnosine as a natural antioxidant and geroprotector: from molecular mechanisms to clinical trials. Rejuvenation Res. 13:156-158; Cassler N. M., Sams R., Cripe P. A., McGlynn A. F., Perry A. B., Banks B. A., Patterns and perceptions of supplement use by U.S. Marines deployed to Afghanistan. Mil Med. 2013 178(6):659-64; Derave, W., et al. (2010). “Muscle carnosine metabolism and β-alanine supplementation in relation to exercise and training.” Sports medicine 40(3): 247-263; Derave W., Ozdemir M. S., Harris R. C., Pottier A., Reyngoudt H., Koppo K., Wise J. A., Achten E., Beta-Alanine supplementation augments muscle carnosine content and attenuates fatigue during repeated isokinetic contraction bouts in trained sprinters. J Appl Physiol 103(5):1736-1743, 2007; Dunnett M., Harris R. C. (1999), Influence of oral beta-alanine and L-histidine supplementation on the carnosine content of the gluteus medius. Equine Vet J Suppl 30:499-504; Del Favoro S., Roschel H., Solis M., Hayashi A., Artiolo G., Otaduy M., Benatti F., Harris R., Wise J., Leite C., Pereira R., de Sa-Pinto A., Lancha-Junior A., Gualano B. (2012), Beta-alanine (Carnosyn™) supplementation in elderly subjects (60-80 years): effects on muscle carnosine content and physical capacity. Amino Acids 43:49-56; Estrada A., Kelley A. M., Webb C. M., Athy J. R., Crowley J. S., Modafinil as a replacement for dextroamphetamine for sustaining alertness in military helicopter pilots. Aviat Space Environ Med. 2012, 83(6):556-64; Evans R. K., Scoville C. R., Ito M. A., Mello R. P., Upper body fatiguing exercise and shooting performance. Mil. Med. 168:451-456, 2003; Gillingham R. L., Keefe A. A., Tikuisis P., Acute caffeine intake before and after fatiguing exercises improves target shooting engagement time. Aviat. Space Environ. Med. 75:865-871, 2004; Green S. B., Salkind N.J., Akey T. M.,: Using SPSS for Windows: Analyzing and Understanding Data (2^{nd} ed.), Upper Saddle River, N.J.: Prentice Hall, 2000; Harman, E. A., et al. (2008). “Effects of two different eight-week training programs on military physical performance.” The Journal of Strength & Conditioning Research 22(2): 524-534; Harris R. C., Tallon M. J., Dunnett M., Boobis L., Coakley J., Kim H. J., Fallowfield J. L., Hill C. A., Sale C., Wise J. A., (2006), The absorption of orally supplied β-alanine and its effect on muscle carnosine synthesis in human vastus lateralis. Amino Acids 30(3)279-289; Hayman M., Two minute clinical test for measurement of intellectual impairment in psychiatric disorders. Arch Neuro Psychiatry 1942; 47:454-64; Hill C. A., Harris R. C., Kim H. J., Harris B. D., Sale C., Boobis L. H., Kim C. K., Wise J. A., (2007), Influence of β-alanine supplementation on skeletal muscle carnosine concentrations and high intensity cycling capacity. Amino Acids 32(2):225-233; Hobson R. M., Saunders B., Ball G., Harris R. C., Sale C., (2012), Effects of Beta-alanine supplementation on exercise performance. a meta-analysis. Amino Acids. 43(1):25-37; Hoffman J. R., Emerson N. S., Stout J. R., β-alanine supplementation. Curr. Sports Med. Rep. 11:189-195, 2012; Hoffman J. R., Ratamess N. A., Faigenbaum A. D., Ross R., Kang J., Stout J. R., Wise J. A., (2008a), Short-duration β-alanine supplementation increases training volume and reduces subjective feelings of fatigue in college football players. Nutr Res 28(1):31-35; Hoffman J. R., Ratamess N. A., Kang J., Mangine G., Faigenbaum A. D., Stout J. R. (2006), Effect of creatine and β-alanine supplementation on performance and endocrine responses in strength/power athletes. Int J Sport Nutr Exerc Metabab 16(4):430-446; Hoffman J. R., Ratamess N. A., Ross R., Kang J., Magrelli J., Neese K., Faigenbaum A. D., Wise J. A. (2008b), β-Alanine and the hormonal response to exercise. Int J Sports Med 29(12):952-958; Jordan T., Lukaszuk J., Misic M., Umoren J., Effects of beta-alanine supplementation on the onset of blood lactate accumulation (OBLA) during treadmill running: Pre/post 2 treatment experimental design. J Int Soci Sports Nutr 7: 20-27, 2010; Kendrick I. P., Harris R. C., Kim H. J., Kim C. K., Dang V. H., Lam T. Q., Bui T. T., Smith M., Wise J. A. (2008), The effects of 10 weeks of resistance training combined with beta-alanine supplementation on whole body strength, force production, muscular endurance and body composition. Amino Acids. 34(4):547-554; Kern B. D., Robinson T. L., Effects of β-alanine supplementation on performance and body composition in collegiate wrestlers and football players. J Strength Cond Res. 2011 25:1804-1815; Knapik, J. J., et al. (2004). “Soldier load carriage: historical, physiological, biomechanical, and medical aspects.” Military medicine 169(1): 45-56; Lieberman H. R., Bathalon G. P., Falco C. M., Kramer F. M., Morgan C. A. 3rd, Niro P., Severe decrements in cognition function and mood induced by sleep loss, heat, dehydration, and undernutrition during simulated combat. Biol Psychiatry. 57:422-429, 2005; Lieberman H. R., Stavinoha T. B., McGraw S. M., White A., Hadden L. S., Marriott B. P., Use of dietary supplements among active-duty US Army soldiers. Am J Clin Nutr. 92:985-995, 2010; Lieberman H. R., Tharion W. J., Shukitt-Hale B., Speckman K. L., Tulley R. Effect of caffeine, sleep loss, and stress on cognitive performance and mood during U.S. Navy SEAL training. Psychopharmacology. 164:250-261, 2002; Murakami T., Furuse M. (2010), The impact of taurine- and beta-alanine-supplemented diets on behavioral and neurochemical parameters in mice: antidepressant versus anxiolytic-like effects. Amino Acids. 39:427-434; Nibbeling, N., et al. (2014). “The effects of anxiety and exercise-induced fatigue on shooting accuracy and cognitive performance in infantry soldiers.” Ergonomics(ahead-of-print): 1-14; Nindl B. C., Barnes B. R., Alemany J. A., Frykman P. N., Shippee R. L., Friedl K. E., Physiological consequences of U.S. army ranger training Med. Sci. Sports Exerc. 39:1380-1387, 2007; Nindl, B. C., et al. (2013). “Physiological Employment Standards III: physiological challenges and consequences encountered during international military deployments.”European journal of applied physiology 113(11): 2655-2672; Nindl, B. C., et al. (2002). “Physical performance responses during 72 h of military operational stress.” Medicine and science in sports and exercise 34(11): 1814-1822; Russo M. B., Arnett M. V., Thomas M. L., Caldwell J. A., Ethical use of cogniceuticals in the militaries of democratic nations. Amer. J. Bioethics. 8:39-49, 2008; Soher, B. J., et al. (1996). “Quantitative proton MR spectroscopic imaging of the human brain.” Magnetic resonance in medicine 35(3): 356-363; Stellingwerff, T., et al. (2012). “Effect of two β-alanine dosing protocols on muscle carnosine synthesis and washout.” Amino acids 42(6): 2461-2472; Stout J. R., Cramer J. T., Mielke M., O'Kroy J., Torok D. J., Zoeller R. F. (2006), Effects of twenty-eight days of β-alanine and creatine monohydrate supplementation on the physical working capacity at neuromuscular fatigue threshold. J Strength Cond Res 20(4)928-931; Stout J. R., Cramer J. T., Zoeller R. F., Torok D., Costa P., Hoffman J. R., Harris R. C., O'Kroy J. (2007), Effects of β-alanine supplementation on the onset of neuromuscular fatigue and ventilatory threshold in women. Amino Acids. 32(3):381-386; Stout J. R., Graves S. B., Smith A. E., Hartmen M. J., Cramer J. T., Beck T. W., Harris R. C. (2008), The effects of beta-alanine supplementation on neuromuscular fatigue in elderly (55-92 years): a double-blinded randomized study. JISSN. 5(21); Van Thienen R., Van Proeyen K., Vanden Eynde B., Puype J., Lefere T., Hespel P., Beta-alanine, improves sprint performance in endurance cycling. Med Sci Sports Exer 41(4):898-903, 2009; Varley, M. C., Fairweather, I. H., and Aughey, R. J., Validity and reliability of GPS for measuring instantaneous velocity during acceleration, deceleration, and constant motion. J Sports Sci 30: 121-127, 2012; Weeks S. R., McAuliffe C. L., DuRussel D., Pasquina P. F., Physiological and psychological fatigue in extreme conditions: The military example. Phys. Med Rehab. 2:438-441, 2010; Wells A. J., Hoffman J. R., Gonzalez A. M., Stout J. R., Fragala M. S., Mangine G. T., McCormack W. P., Jajtner A. R., Townsend J. R., and Robinson I V, E. H., A Supplement Containing Phosphatidylserine Attenuates Post-Exercise Mood Disturbance and Perception of Fatigue in Humans. Nutrition Research. 33:464-472, 2013; Welsh T. T., Alemany J. A., Montain S. J., Frykman P. N., Young A. J., Nindl B. C., Effects of intensified military field training on jumping performance. Int. J. Sports Med. 29:45-52, 2008.

### SUMMARY OF THE INVENTION

Embodiments of the present invention provide methods for maintaining or improving tactical and/or psychomotor functions after highly intense and fatiguing activities.

Embodiments of the present invention may include methods for maintaining or improving tactical and/or psychomotor functions before, during, and/or after highly intense and fatiguing activity. In one embodiment, this is achieved by supplementing the human diet with an effective amount of the free amino acid beta-alanine, or a salt or ester thereof, wherein the effective amount is provided over a period of time. Naturally, a salt or ester of beta-alanine could be taken and would readily convert to the free amino acid in the body or in a suitable delivery medium. In some embodiments of the present invention the free amino acid beta-alanine, or a salt or ester thereof can be administered before, during or after the tactical and/or psychomotor functions. In other embodiments of the present invention the free amino acid beta-alanine, or a salt or ester thereof can be administered before, during or after the highly intense and fatiguing activity.

Embodiments of the present invention include methods for maintaining or improving psychomotor performance before, during and/or after highly intense and fatiguing activity. In one embodiment, this is achieved by supplementing the human diet with an effective amount of the free amino acid beta-alanine, or a salt or ester thereof, wherein the effective amount is provided over a period of time.

Other embodiments of the present invention provide methods for improving or maintaining lower body power and performance before, during and/or after highly intense and fatiguing activity. In one embodiment, this is achieved by supplementing the human diet with an effective amount of the free amino acid beta-alanine, or a salt or ester thereof, wherein the effective amount is provided over a period of time. In some embodiments of the present invention the free amino acid beta-alanine, or a salt or ester thereof can be administered before, during or after the highly intense and fatiguing activity.

Other embodiments of the present invention provide methods for increasing the distance run at high velocity. In some embodiments the distance run at high velocity is a part of a larger distance run at a mixture of high, moderate and low velocity, such as a percentage of a larger distance. For example the distance run at high velocity may be a percentage of a larger distance. In one embodiment, this is achieved by supplementing the human diet with an effective amount of the free amino acid beta-alanine, or a salt or ester thereof, wherein the effective amount is provided over a period of time. In some embodiments of the present invention the free amino acid beta-alanine, or a salt or ester thereof can be administered before, during or after the highly intense and fatiguing activity.

Other embodiments of the present invention provide methods of enhancing the effectiveness of units, such as military units or sports teams. In some embodiments, enhancing the effectiveness of these units includes issuing individuals in the unit a human dietary supplement comprising an effective amount of the free amino acid beta-alanine, or a salt or ester thereof, wherein the effective amount is provided over a period of time. In some embodiments, the step of issuing individuals in the unit a human dietary supplement also includes instructions on the use of the human dietary supplement. In some embodiments, the step of issuing individuals of the unit a human dietary supplement is performed by a person authorized to do so by the sports team, such as a manager, a team doctor, a sports nutritionist and the like. In some embodiments, the step of issuing individuals of the unit a human dietary supplement is performed by a person authorized to do so by the military unit, such as a commanding officer, a doctor, a training manager and the like. In some embodiments, enhancing the effectiveness of these units includes improving passing accuracy during and after physical exertion; improving shooting accuracy during and after physical exertion; increasing psychomotor function; decreasing reaction time; decreasing involuntary muscle action or movement caused by physical stress during critical actions; increasing control of breathing or breath during and after physically fatiguing and stressful situations; or combinations thereof.

The period of time over which the effective amount described herein of beta-alanine is provided is about 7 days or more. Additionally, the beta-alanine can be given every day over this period of time, may be given on alternative days, or given periodically over this period of time.

Also, the present invention provides methods for avoiding physical and psychomotor decrements related to intense and sustained actions, such as those associated with military actions and others as described herein.

Additional features, advantages, and embodiments of the invention are set forth or apparent from consideration of the following detailed description, drawings and claims. It is to be understood that both the foregoing summary of the invention and the following detailed description are exemplary and intended to provide further explanation without limiting the scope of the invention as claimed.

### BRIEF DESCRIPTION OF THE DRAWINGS

FIG. 1 shows a potential study design involving one possible supplementation and testing protocol to demonstrate the maintenance or improvement of tactical performance and/or psychomotor performance after fatiguing activity.

FIG. 2 is a schematic design of one study protocol.

FIG. 3a shows Vertical Jump Relative Peak Power Performance.

FIG. 3b shows Vertical Jump Mean Power Performance (*=significant difference between groups).

FIG. 4a shows Shooting Accuracy.

FIG. 4b shows Time per Shot on Target (*=significant difference between groups).

FIG. 5 shows Serial Subtraction test.

FIG. 6 shows changes in Δ carnosine content in the gastrocnemius. All data are reported as mean±SD. *=Significant difference (p<0.05) between groups; BA=β-alanine, PL=placebo.

FIG. 7 shows Spearman rho Correlation between changes in muscle carnosine content and fatigue rate in the 1-min Sprint.

FIG. 8 shows Spearman rho Correlation between changes in muscle carnosine content and changes in 50-m Casualty Carry.

FIG. 9 shows changes in Δ 50-m Casualty Carry. All data are reported as mean±SD. *=Significant difference (p<0.05) between groups; BA=β-alanine, PL=placebo.

FIG. 10 shows changes in Serial Subtraction Test. All data are reported as mean±SD. *=Significant difference (p<0.05) between groups; BA=β-alanine, PL=placebo.

### DETAILED DESCRIPTION OF THE PREFERRED EMBODIMENTS

Methods and embodiments are described for maintaining or improving tactical performance and/or psychomotor performance function through dietary supplementation with the free amino acid beta-alanine, or salt or ester thereof, and are for illustrative purposes only. The methods described herein may be used for many different industries, including, for example, military, paramilitary organizations, first responders, such as firemen and ambulatory personnel, emergency and surgical personnel, sports teams and many others. The following provides further description of certain embodiments of the invention. As described and claimed here, certain terms are defined and used interchangeably.

As used herein, “β-alanine”, “beta-alanine”, and “BA” are meant to represent the amino acid beta-alanine that is a free amino acid, or a salt or ester of the free amino acid. Unless specified otherwise herein, the use of these interchangeable terms does not encompass beta-alanine as a component of a dipeptide, oligopeptide, or polypeptide. Consequently, a human dietary supplement containing a dipeptide, oligopeptide, or polypeptide without any free amino acid beta-alanine, or an ester or salt thereof, would not be within the scope of the present invention. For example, a dietary supplement of carnosine, or the like, without any free amino acid beta-alanine, would not be within the scope of the present invention. If, however, a human dietary supplement comprises a dipeptide, oligopeptide, or polypeptide in combination with the free amino acid beta-alanine, or an ester or salt thereof, then such human dietary supplement would be within the scope of the present invention, provided the free amino acid beta-alanine, or an ester or salt thereof, is present in an effective amount as defined herein. Naturally, the ester and amide forms of the free amino acid beta-alanine, and their salts, could be used in a similar manner, although those forms are not in these originally submitted claims. Additionally, the use of these interchangeable terms in describing the human dietary supplement of the invention does not encompass beta-alanine from a natural or conventional food or food product unless otherwise specifically stated or claimed. Natural or conventional foods or food products include, but are not limited to, beef, pork, chicken, meat extract supplements, and predigested meat/protein supplements, and the various essences of meats. Under these definitions, the term “human dietary supplement” does not encompass, and does not mean, a natural or conventional food or food product, such as chicken meat, meat essences, chicken broth or meat flavoring. Furthermore, human dietary supplements of the present invention do not encompass pharmaceutical compositions, and the methods of the present invention do not encompass therapeutic treatments. As will be understood, these terms are to be used interchangeably except as otherwise specified herein.

As used herein, the term “human dietary supplement” is intended to mean a dietary supplement as defined under the Dietary Supplement Health and Education Act of 1994 (“DSHEA”). A human dietary supplement as used herein, also means a dietary supplement that is administered or taken by an individual more than once with the purpose of supplementing the diet to increase and/or maintain a component (e.g., beta-alanine) of the supplement, or a substance comprising a component of the supplement (e.g., carnosine) in the body at a higher level(s) than that naturally occurring through natural or conventional meals. Additionally, human dietary supplement further means an addition to the human diet in a pill, capsule, tablet, powder, or liquid form, which is not part of a natural or conventional food or food product, and which effectively increases the function of tissues when consumed.

As used herein, the term “period of time”, “over time” or “duration of time” means more than a single dosing, taking or administration of the human dietary supplement. More specifically, these terms mean the human dietary supplement is taken one or more times per day over a period of seven or more days, wherein generally no two consecutive days pass without the dietary supplementation and the individual supplements the diet at least 3 or 4 days in any 7 day period, more preferably 4 or more days in any 7 day period, more preferably 5 or more days in any 7 day period, more preferably 6 or more days in any 7 day period, more preferably 7 consecutive days in any 7 day period. For example, the individual can take the dietary supplement every day, wherein the dietary supplement is provided over the course of the day or the individual may take a single dose of the dietary supplement. The individual may also account for non-supplementation days as described above regarding days without supplementation. The period of time described herein can be continued for at least 7 days to about 240 days; preferably about 14 days to about 210 days; more preferably about 21 days to about 180 days; more preferably about 28 days to about 180 days; even more preferably about 28 days to about 60 days. It will be understood by those of skill in the art, that the period of time can be adjusted by the individual depending on the desired level of performance to be achieved and/or maintained.

As used herein, the term “effective amount” or “amount effective to” refers to an amount of the supplement required to achieve the increases or improvements sought and is an amount that is more than contained in the average diet. For example, omnivores consume about 50-300 mg of carnosine per day and the cooking procedures used would lead to a beta-alanine amount lower than this. It will be understood by those skilled in the art that a one time, single dosing of beta-alanine is incapable of achieving an effective amount for the purposes of dietary supplementation with beta-alanine. Furthermore, it will be understood by those skilled in the art that administering a single dose followed with multiple consecutive days of non-dosing or non-supplementation will not achieve the effective amount as described in the invention.

As used herein, the term “tactical performance” refers to, but not limited to, performing operational tasks. For example, in a military, paramilitary or police action settings and training, this can include the efficient and accurate handling and operation of explosives, explosive devices, various weapons systems and devices, such as automatic and semi-automatic rifles and handguns, as well as other equipment often employed in such military, paramilitary or police action settings and training. This includes decision making capabilities under stressful and physically stressful situations and periods of time often associated with these fields of work. For first responder action, this can include, but not limited to, the efficient and accurate use of firefighting equipment and machinery, and life-saving medical equipment including drug administration and wound treatments, for example.

As used herein, the term “mental stress”, “mentally stressful” or “stressful environment” refers to situations where the individual(s) is experiencing intense decision making scenarios, especially relating to life and death situations, e.g., combat, controlling crowds and riots, being engaged in emergency response activities, and making decisions on troop deployments for enemy and target engagement. Such mental stresses are compounded in areas of work where, for example, sleep is limited or infrequent, meals are irregular, excessive and/or continued physical exertion is required and there is a frequent state of physical and mental fatigue being experienced. In combat, the intersection of mental and physical stress is referred to as the “fog of war.”

As used herein, the term “psychomotor performance” or “psychomotor function” refers to, but not limited to, the coordination of a sensory or cognitive process and a motor activity as demonstrated by a subject through the completion of a task. For example, this can be demonstrated by a person required to acquire a target, engage the target effectively and accurately, and effectively address problems during target engagement. This psychomotor performance is also present in many sports such as soccer, football, hockey and rugby in which individuals experience highly intense and fatiguing activity and must also coordinate a sensory or cognitive process and a motor activity to complete a task, such as accurately passing to a team mate, or accurately shooting towards the goal. Such psychomotor performances or functions are relevant before, during and/or after highly intense and fatiguing activity.

As used herein, the term “run at high velocity” is intended to mean run at speeds that are higher than the average jogging speed. For example, these speeds can be in excess of 4.4 m·sec⁻¹. Also, the total distance run, which includes the distance run at high velocity in addition to the distance run at low and moderate velocities, e.g., average jogging speed, can be a distance that is generally considered “middle distance.” As used herein, the term “middle distance” is intended to mean a distance from about 800 meters to about 10,000 meters. This distance can be performed all at one time, such as in a 4 km run or, e.g., a 5,000 meter race at a track and field competition, or throughout the period of another activity, such as a tactical training exercise, or, e.g., a soccer game.

Many factors affect marksmanship, which requires coordination of cognitive processes and motor activities, such as deciding on, sighting in, and engaging a target. During activities such as combat, combat training or simulations, and police or paramilitary actions, individuals required to engage targets can be placed under severe physical and mental stress that can greatly affect psychomotor function. Because physical and mental stress can affect heart rate, mental acuity, physical body control, and decision making processes related to target engagement and marksmanship, it is paramount for an individual in such situations to be as focused and calm as possible if the need to obtain and engage a target arises. For example, the ability to acquire a target and properly maintain sight alignment with the weapon to insure maximum accuracy requires a total state of physical and mental awareness of the situation and one's own body. This includes one's ability to control breathing and maintain posture control while sighting in on a target. Breathing causes the individual's torso (upper body) to expand and contract, which in turn moves other parts of the body that can alter the firing posture, thereby causing temporary loss of target impact zone. Physical or mental exhaustion or fatigue may also lead to a diminished focus on firing posture during target engagement. Because breathing and failed posture can cause unwanted movement when attempting to engage a target, it is important for the individual to control breathing and maintain firing posture while accurately sighting in and engaging the target. Therefore, being able to improve or maintain proper breathing and posture control during physically and mentally stressful situations, such as combat, can increase the individual's ability to effectively and accurately acquire and engage a target. This is often the situation for combat soldiers who are required to move, for example, repeatedly from one location to the next while acquiring and engaging targets and maintaining full situational awareness as part of the decision making process. The present invention provides methods for improving or maintaining times for target acquisition, accuracy and engagement by improving or maintaining an individual's ability to control breathing and firing posture during times of need as described herein.

The invention provides an important understanding of how a nutrient or dietary supplement provided over a period of time can maintain or improve tactical performance and/or psychomotor performance before, during and after highly intensive and physically and mentally fatiguing activities, e.g., military performance and tactical functions. The invention also provides methods for maintaining or improving special tactical and strategic performance in military, paramilitary and first responders.

### Forms and Formulations

Administration of the beta-alanine can be as the free amino acid beta-alanine, wherein the free amino acid is not part of a dipeptide, oligopeptide or polypeptide. The free amino acid can be an ester or salt of beta-alanine. The free amino acid can be in a pill, tablet, capsule, granule or powder form. The free amino acid can be administered as part of a solid, liquid or semi-liquid. The free amino acid can be administered as part of a drink (e.g., sports drink) or a food (e.g., health bar).

The beta-alanine may also be administered in a sustained release formulation, wherein the free amino acid beta-alanine is not part of a dipeptide, oligopeptide, or polypeptide. The beta-alanine administered in a sustained release formulation may also be present as an ester or salt of the beta-alanine. The sustained release formulation can be in a tablet, capsule, granule or powder form. The sustained release formulation can be administered as part of a solid, liquid or semi-liquid. The sustained release formulation of the free amino acid beta-alanine can be administered as part of a drink (e.g., sports drink) or a food or food matrix (e.g., health or energy bar or energy gel). It has been reported that some individuals may experience a slight flushing/tingling of the skin when taking β-alanine as a free amino acid. While this sensation may be uncomfortable, it typically lasts less than 60 minutes. The use of sustained-release forms of beta-alanine has been shown to inhibit, decrease and/or eliminate the flushing/tingling of the skin.

In various embodiments of the present invention, the human dietary supplement may be administered (e.g., consumed or ingested) in combination with other ingredients. For example, the free amino acid beta-alanine, or an ester or salt thereof, may be administered in combination with creatine, wherein the creatine is in the form of creatine-monohydrate or other acceptable forms of creatine. Creatine is desirable due to the enhanced ergogenic effect of the formulations of the current invention.

In another embodiment, the dietary supplement comprising the free beta-alanine can further comprise one or more carbohydrates, including simple carbohydrates, for example. Additionally, carbohydrates can include starch and/or sugars, e.g., glucose, fructose, galactose, sucrose, and maltose. The sugars or other carbohydrates can be from various forms of honey, molasses, syrup (e.g., corn syrup, glucose syrup), treacle or gels. It will be understood that the human dietary supplement of the invention may comprise one or more carbohydrates in combination with the other ingredients disclosed herein and as part of the forms and formulations defined by the present invention.

In addition, the human dietary supplements of the present invention may further comprise insulin, insulin mimics, and/or insulin-action modifiers. Insulin mimics include, but are not limited to, D-pinitol (3-O-methyl-chiroinositol), 4-hydroxy isoleucine, L783,281 (a demethyl-asterriquinone B-1 compound), alpha lipoic acid, R-alpha lipoic acid, guanidiniopropionic acid, vanadium compounds such as vanadyl sulfate or vanadium complexes such as peroxovanadium, and synthetic phosphoinositolglycans (PIG peptides). Insulin-action modifiers that enhance or inhibit the action of insulin in the body, can include, but are not limited to, sulphonylureas, thiazolidinediones, and biguanides. Additionally, the human dietary supplements may comprise insulin stimulating agents (e.g., glucose).

In another embodiment, the dietary supplement comprising the free beta-alanine can further comprise one or more electrolytes and/or vitamins (e.g., vitamins B6, B12, E, C, and thiamin, riboflavin, niacin, folic acid, biotin and pantothenic acid). In other embodiments, the human dietary supplement may comprise lipids, other amino acids, fiber, trace elements colorings, flavors, natural and/or artificial sweeteners, natural health improving substances, anti-oxidants, stabilizers, preservatives, and buffers.

In certain other embodiments, the human dietary supplement of the present invention may comprise other ingredients, for example, anti-oxidants, alpha-lipoic acid, tocotrienols, N-acetylcysteine, co-enzyme Q-10, extracts of rosemary such as carnosol, botanical anti-oxidants such as green tea polyphenols, grape seed extract, COX-1 type inhibitors such as resveratrol, ginkgo biloba, pterostilbene and garlic extracts. Other amino acids such as L-histidine, L-cysteine and/or L-citrulline may be added. In some embodiments, the present invention may comprise combination with an acetylcholine precursor such as choline chloride or phosphatidylcholine may be desirable, for example, to enhance vasodilation. The invention also provides for human dietary supplements comprising the free amino acid beta-alanine in combination with such other ingredients as minerals and trace elements in any type or form suitable for human consumption. It is convenient to provide calcium and potassium in the form of their gluconates, phosphates or hydrogen phosphates, and magnesium as the oxide or carbonate, chromium as chromium picolinate, selenium as sodium selenite or selenate, and zinc as zinc gluconate.

The ingredients, compounds and components disclosed herein as optionally being in the human dietary supplement comprising the free amino acid beta-alanine, may be in any combination as part of the human dietary supplement. This will be readily understood by those of skill in the field of dietary supplementation and exercise physiology.

Once the levels of beta-alanylhistidine have been increased by use of effective amounts of the human dietary supplement, otherwise known as a loading phase, the dosing can be adjusted to maintain the levels of beta-alanylhistidine necessary to maintain or improve tactical performance and/or psychomotor performance for the purposes of this invention.

The forms and formulations, provided herein, can be those as described and provided for in U.S. Pat. Nos. 5,965,596, 6,426,361, 7,825,084, 8,067,381, and 8,329,207, each of which is incorporated by reference in its entirety.

In one aspect, the dietary supplement is formulated for one or more servings that can be ingested one or more times per day to achieve an effective amount as required by the present invention. Thus, the total daily intake amount required to meet an effective amount of free beta-alanine, or an ester or salt thereof, can be obtained through a single serving or through multiple smaller servings throughout the day that in total meet the required amount of free beta-alanine, or an ester or salt thereof, to be an effective amount in a total daily intake of the dietary supplement. Therefore, a dietary supplement can be formulated with lower amounts of free beta-alanine, or an ester or salt thereof, for the purpose of multiple servings in a day, wherein the total amount through multiple servings meets the desired total daily intake to be an effective amount as defined by the present invention.

The total daily intake amount of the free amino acid beta-alanine, or an ester or salt thereof, is in a range of about 0.3 grams (g) to about 16.0 g; preferably about 1.0 g to about 10.0 g; more preferably about 2.0 g to about 8.0 g; and even more preferably about 3.0 g to about 7.0 g. As described in the present invention, the total daily intake amount in these ranges can be achieved through a single serving formulation comprising the desired effective amount of free beta-alanine. Alternatively, the total daily intake amount in these ranges can be achieved through a formulation for multiple servings, each comprising an amount of the free beta-alanine that when totaled for the day will be within the desired range for a total daily intake delivering an effective amount as defined by the present invention.

Where the dietary supplement is formulated for multiple servings per day within the ranges described herein, it will be understood that there can be 2-12 servings or more, depending on the amounts of free beta-alanine, or ester or salt thereof, in the formulated units. For example, a sustained release tablet comprising 2.0 g of free beta-alanine can be served 3 times per day for a total daily intake of 6.0 g of free beta-alanine. As another example, a formulation comprising 0.5 g of free beta-alanine can be taken 12 times throughout the day for a total daily intake of 6.0 g. This aspect of the present invention applies whether 12 tablets comprising 0.5 g of free beta-alanine are taken at 12 different times throughout the day or if 4 tablets are taken at 3 different times throughout the day. As will be understood in the present invention, it is the total daily intake of the free beta-alanine that must be an effective amount as defined by the present invention. Moreover, the effective amounts in the ranges provided herein account for non-supplementation days as defined by the present invention. Therefore, as long as the individual supplements his/her diet as described herein, the total daily intake of the dietary supplement accounts for non-supplementation days and achieves an effective amount as required over time.

These ranges for total daily intake of free beta-alanine can also account for the various body sizes. Therefore, it will be understood that individuals with smaller body types can take less or more depending on the desired performance levels to be achieved. Likewise, the ranges for total daily intake account for individuals with larger body types that might require a higher total daily intake to achieve the desired performance levels. Regardless of body type, the total daily intake of the effective amounts of free beta-alanine, or an ester or salt thereof, in the dietary supplements of the present invention can account for adjustments in amounts based on the individual's body type requirements and desired performance levels.

It will also be understood that an effective amount being consumed, as defined herein, can be adjusted up or down as long as the total daily intake of free beta-alanine, or an ester or salt thereof, is maintained within the ranges provided herein and meet the definition of effective amounts of the present invention. For example, an individual taking a dietary supplement of the present invention in a formulation delivering a total daily intake of 6.0 g or 6.4 g, can adjust the level of supplementation down to 1.6 g, 2.0 g, 3.0 g or 3.2 g of a total daily intake of the free amino acid beta-alanine, or an ester or salt thereof. This is referred to as a maintenance phase. It will be understood by those of skill in the art that individuals may reach a desired level of performance through the dietary supplementation of the present invention and then the individual can opt to reduce the effective amount to a lower effective amount of the present invention to maintain the level of performance achieved. In a converse example, an individual taking a total daily intake of 3.0 g or 3.2 g of the free beta-alanine, or an ester or salt thereof, as an effective amount, can increase the total daily intake of the free beta-alanine to any effective amount within the ranges described herein. For example, the individual could increase the total daily intake from 1.6 g, 2.0 g, 3.0 g or 3.2 g to a total daily intake of 6.0 g or 6.4 g, if a further increase in performance is desired. These examples of adjusting the total daily intake of the free beta-alanine described herein are intended as examples of how an individual can increase the level of performance or maintain an achieved level of performance, and these examples are not intended to be limiting on the present invention.

It will also be understood from the present disclosure that an individual can cycle the intake of an effective amount of the free beta-alanine between higher and lower total daily intakes of an effective amount of beta-alanine. For example, an individual could take a total daily amount of 6.4 g of the free beta-alanine, or an ester or salt thereof, as an effective amount for a period of 28 days, including non-supplementation days, followed by 28 days of taking 1.6 g of the free beta-alanine, or an ester or salt thereof, as an effective amount, including non-supplementation days, followed by 28 days of taking 6.4 g of the free beta-alanine, or an ester or salt thereof, as an effective amount, including non-supplementation days. It will also be understood that the time periods and total daily intake amounts given in the example of cycling can be adjusted based on the individual's body type requirements and desired performance levels.

As will be understood by one of skill in the art through the disclosure of the present invention, other ingredients, e.g., creatine, other amino acids, and carbohydrates, can be present in the human dietary supplement in similar amounts as that described for the free amino acid beta-alanine, or esters or salts thereof.

Given the potential for prolonged deployments in combat zones, extended training missions, military-type field exercises, and training academies and similar events occurring over extended periods of time, it may be preferred that the forms and formulations of the human dietary supplement described herein be provided or issued in conjunction with meals manufactured for such settings. For example, the U.S. military provides field rations, in the form of Meals-Ready-To-Eat (“MREs”), to soldiers as a sustaining source of nutrition. Therefore, the forms and formulations of the human dietary supplement of the present invention may be supplied as an additional component of the field rations through a human dietary supplement packet or kit for consumption in accordance with the present invention. The forms and formulations of the human dietary supplement of the present invention may also be incorporated as an ingredient in the actual food as packaged in the field rations. Offering the human dietary supplement of the present invention as a packet or kit in the field rations or as an ingredient in the actual food of the field rations will enable the personnel to continue the dietary supplementation thereby maintaining or improving their tactical performance and/or psychomotor performance. This permits personnel in the military unit, other similarly defined units, and the units as a whole to maintain a level of preparedness and readiness required in combat and police actions, for example. These units can have improved shooting accuracy, decreased reaction time to relevant situations, decreased involuntary muscle action or movement caused by physical stress in critical actions, increased control over firing posture, increased control of breathing or breath during and after physically fatiguing and stressful situations, or combinations of these benefits. This level of improved or enhanced readiness is advantageous to the tactical performance and/or psychomotor performance of the individuals in the unit and the unit as a whole.

As discussed in the present invention and demonstrated in the examples below, increases in time for tactical and/or psychomotor functions and responses are paramount in certain fields of work and can be crucial in life-threatening and/or lifesaving scenarios, such as those faced in military and police actions, and medical and fire emergencies. Therefore, the present invention offers the units and individuals within these fields of work a method of supplementing the diet to improve and/or maintain their operational awareness and effectiveness.

### Study 1

### Examples

In one embodiment, members of a military special operations unit can be given the human dietary supplement and tested for muscular performance, tactical performance, psychomotor performance and cognitive function. In one embodiment the subjects can be between 21-39 years old, be free of any physical limitations (determined by health and activity questionnaire), and not using or have taken any nutritional supplement for the past 30 days.

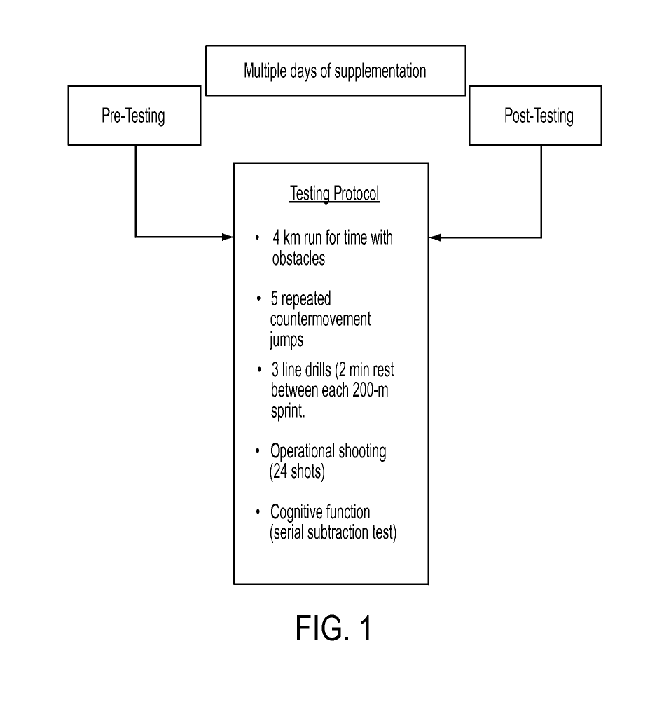

Data collection can occur on two separate occasions that are separated by 28 days, but shorter periods, such as 7 days, and longer periods, such as 56 days or more, are also encompassed by the present invention. FIG. 1 provides a schematic design of one embodiment of a protocol. For example, subjects perform a 4 km run with normal operational load and weapon (˜20 kg). During the run, subjects overcome several obstacles such as wall climb, rope climbs and other potential barriers. At the end of the 4 km run subjects perform five countermovement jumps and then proceed to run three 200-m shuttles (line drills) with a 2-min rest between each sprint. Following the last sprint subjects run to the shooting range and perform shooting protocols with their weapon. Finally, subjects complete a serial subtraction test to assess cognitive function in a fatigued state. All subjects perform the first trial prior to supplementation and the second trial following supplementation. As noted above, the supplementation period can be 7 days or more, with the supplementation occurring on at least 3 or 4 days in any 7 day period, more preferably 4 or more days in any 7 day period, more preferably 5 or more days in any 7 day period, more preferably 6 or more days in any 7 day period, and more preferably 7 consecutive days. All subjects are provided with an individual global positioning system (GPS) that they wear in a vest underneath their uniform. The GPS unit (MinimaxX, V4.3, Catapult Innovations, Victoria, Australia) is positioned in a posterior pocket on the vest situated between the subject's right and left scapula in the upper-thoracic spine region. Information on velocity and distance of activity is recorded during the 4 km run and repeated sprints. In addition, all gravitation forces (G force) in the G_{Z}, G_{X}, G_{Y} planes of movement are measured. The G forces that accumulate during the course of each activity will be defined as the Subject Load. Subject load is an accumulated rate of change of acceleration calculated with the following formula:

$$Subject Load = {\sum }_{t = 0}^{t = n} \sqrt{( {( {fwd}_{t = i + 1} - {fwd}_{t = i} )}^{2} + {( {side}_{t = i + 1} - {side}_{t = i} )}^{2} + {( {up}_{t = i + 1} - {up}_{t = i} )}^{2} )}$$

Where: Fwd=forward acceleration; side=sideways acceleration; up=upwards acceleration; i=present time; t=time.

Data is collected at 10 Hz and analysis can be performed with the system software provided by the manufacturer.

To quantify vertical jump power, subjects can perform five consecutive countermovement jumps (CMJ). During each jump subjects stand with hands on the waist at all times. The subjects are instructed to maximize the height of each jump while minimizing the contact time with the ground between jumps. During each jump the subject wears a belt connected to a Tendo™ Power Output Unit. The average peak and mean power outputs for all five jumps are recorded.

Following the final sprint, subjects run to the shooting range and shoot 4 shots per target at 6 meters, 12 meters and 25 meters. For the first two targets, subjects use a handgun, while for the final target subjects use an assault rifle. The second series of shooting occurs using similar target ranges, but requires the subject to identify friend from foe during each shooting attempt.

Cognitive function can be analyzed by various methodologies. For example, a modified version of the original Serial Sevens Test can be utilized to analyze cognitive function. One version of this test consists of a two minute timed oral test in which individuals are required to subtract the number 7 from a random computer generated four digit number, to measure how quickly and accurately the individual can compute a simple mathematical problem. The computer generated numbers can be written onto standard note cards. Individuals can be given a randomized stack of note cards and asked to complete as many calculations as possible in the two minute period. The individual and scorer can sit opposite each other during testing. The answers to the calculations can be written on the back of the note cards in pencil for the scorer to see. Individuals should not see the correct answer. Once the individual releases the note card, the answer is considered unchangeable. The number of correct answers and the average time per correct answer is recorded.

In consideration of the known qualities of β-alanine supplementation, and the potential neurological effects, there appears to be a potential benefit for it to be used as a dietary supplement in preparation for prolonged, high intense military activity that requires maintaining high levels of physical performance, focus, and decision making ability under stressful and fatiguing conditions.

Twenty male soldiers from a special operations unit of the Israel Defense Force (IDF) volunteered to participate in this study to examine the effect of 28 days of beta-alanine ingestion on physical, tactical, psychomotor and cognitive performance in military personnel. Following an explanation of all procedures, risks and benefits, each participant provided his informed consent to participate in the study. The Helsinki committee of the IDF Medical Corps approved the research protocol. Subjects were not permitted to use any additional nutritional supplementation and did not consume any androgens or any other performance enhancing drugs known to increase performance. Screening for performance enhancing drug use and additional supplementation was accomplished via a health questionnaire completed during participant recruitment. All participants were from the same unit, but were from three different squads. Volunteers from each squad were randomly assigned to one of two groups. The first group; (BA; 20.1±0.7 years; height: 1.79±0.07 m; weight: 78.3±9.7 kg) consumed 6.0 g of β-alanine per day, while the second group (PL; 20.2±1.1 years; height: 1.80±0.05; weight: 79.6±7.8 kg) consumed 6 g of placebo (rice flour). There were an equal number of participants from each squad that were randomly assigned to either BA or PL.

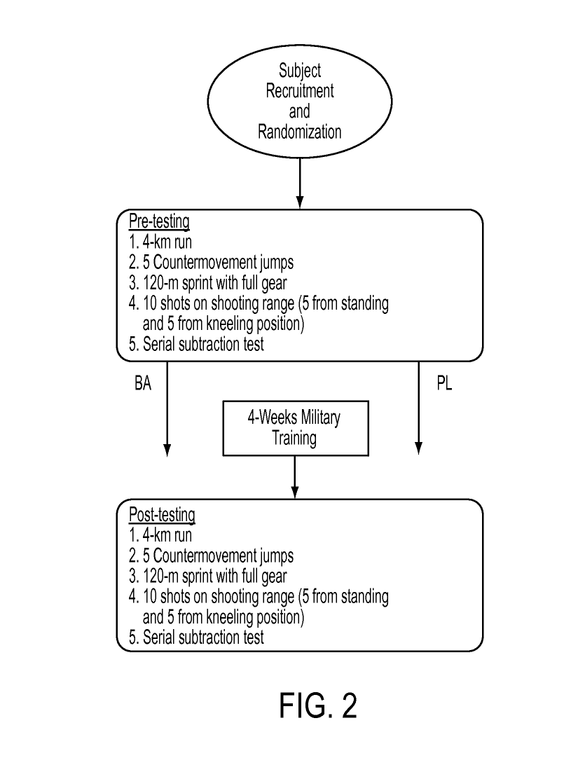

The study was conducted at the unit's training facilities, under the unit's regular training protocols and safety regulations. Data collection occurred on two separate occasions that were separated by 28 days. FIG. 2 provides a schematic design of the study protocol. Each session required all participants to perform a 4 km run dressed in shorts, T-shirt and running shoes. Immediately following the 4 km run, participants performed five countermovement jumps. Participants then proceeded to put on their operational gear and weapon and ran a 120 m sprint. Following the sprint, participants proceeded as quickly as possible onto the shooting range and performed a 10-shot shooting protocol with their assault rifle. Five shots were performed in the standing position, and five shots were performed in the kneeling position. During the shooting a planned misfire occurred that required the participant to correct and resume shooting. Immediately following the shooting drill all participants completed a 2-minute serial subtraction test to assess cognitive function in a fatigued state.

All participants were provided with an individual global positioning system (GPS) that they wore in a vest underneath their shirt. The GPS unit (MinimaxX, V4.3, Catapult Innovations, Victoria, Australia) was positioned in a posterior pocket on the vest situated between the participant's right and left scapula in the upper-thoracic spine region. Information on velocity patterns was recorded during the 4 km run. Peak velocity, mean velocity, distance covered running at slow-moderate speed (<4.44 m·sec-1), distance covered running at high speed (4.44+m·sec-1), and the percent of total distance run at slow-moderate and high speeds were downloaded from the GPS receiver/transmitters. Data was collected at 10 Hz and all analysis was performed with the system software provided by the manufacturer. The validity and reliability of the GPS technology has been previously demonstrated (Varley et al., 2012).

Jump Power

To quantify vertical jump power, participants performed five consecutive countermovement jumps (CMJ). During each jump participants stood with their hands on their waist at all times. The participants were instructed to maximize the height of each jump while minimizing the contact time with the ground between jumps. During each jump the participant wore a belt connected to a Tendo™ Power Output Unit (Tendo Sports Machines, Trencin, Slovak Republic). The Tendo™ unit consists of a transducer attached to the end of the belt that measured linear displacement and time. Subsequently, the velocity of each jump was calculated and power determined. The average peak and mean power outputs for all five jumps were recorded. Test-retest reliability for the Tendo™ unit in our laboratory has consistently shown R>0.90.

Shooting Performance

Targets were set at a 40-m distance from the firing line and were all headshots. Each shot that hit the target was considered accurate. Twenty targets were set up on the range. All participants were notified prior to the start of data collection which target they were required to shoot at. Immediately following the 120-m sprint, participants continued onto the shooting range and shot 5 times while standing and 5 times from a kneeling position with their assault rifle. Participants were requested to shoot rapidly and accurately. While shooting, each participant was required to handle a misfire in their weapon. The misfire was prearranged by the investigative team, which involved placing an empty bullet into weapon's magazine (weapon's ammunition storage and feeding device). This required the participant to recognize and correct the misfire and continue to deliver fire at the designated target. The designed misfire was set to increase the stress of the shooting, with the participants already fatigued from the 4 km run, jumps and sprint with full gear. The number of accurate shots and the time required to perform these shots was recorded.

Cognitive Function

A modified version of the original Serial Sevens Test (Hayman, 1942) was employed to analyze cognitive function. The test consisted of a two minute timed written test in which participants were required to subtract the number 7 from a randomly generated four digit number, in order to measure how quickly and accurately they can compute a simple mathematical problem. The four digit number appeared on the top of the first column of a three column sheet of paper. Participants were provided the sheet of paper and asked to complete as many calculations as possible in the two minute period. Participant and timer/scorer sat opposite each other during testing. The answers to the calculations were written underneath the initial number. Regardless of answer provided, participants were then required to subtract the number 7 from that new number. Participants were not told if their answer was correct or not. The number of correct answers was recorded. Intraclass correlations for this assessment has been determined in our laboratory to be R>0.81 (Wells et al., 2013).

Supplement Schedule

The β-alanine supplement (CarnoSyn™) was obtained from Natural Alternatives International (San Marcos, Calif., USA). Both the supplement and placebo were in tablet form and were similar in appearance. Participants in the supplement group were provided with 2 tablets of sustained-release β-alanine at a dose of 2 g per serving three times per day (total β-alanine intake was 6 g per day) and subjects in the placebo group were provided with an equivalent amount of rice powder. Participants were instructed to consume the supplement separately from their meals. Each participant was provided with a bottle containing a week's supply of tablets. All bottles were returned at the end of the week. All tablets left in the bottle were counted, recorded, and the next week's bottle was provided to the participant. Supplementation occurred over a 28 day period.

Statistical Analysis

Data were analyzed using a 2×2 [treatment (BA, PL) X time (pretest, posttest)] mixed factorial ANOVA. Differences in the mean posttest performance values were determined by using analysis of covariance, with pretest values serving as the covariate. One-Way Analysis of Covariance (ANCOVA) was utilized to analyze differences between treatment groups. For effect size (ES), the partial eta squared statistic was reported, and according to Green et al. (2000), 0.01, 0.06, and 0.14 represents small, medium, and large effect sizes, respectively. An alpha level of p<0.05 was used to determine statistical significance. Data was analyzed using SPSS v20 software (SPSS Inc., Chicago, Ill.).

Results

Compliance for consuming the supplement or placebo was 97%. During the 4-week training period, the decrease in body mass in BA (−1.3±1.0 kg) was significantly greater (p=0.14, ES=0.34) than PL (−0.2±0.6 kg). Comparison of performance measures between BA and PL during the 4-km run can be seen in Table 1. When collapsed across groups, a significant increase (p=0.019) in time for the 4-km run was observed from pre-supplementation (“Pre”) to post-supplementation (“Post”) in both groups combined. No significant interactions were noted, however, between the groups (p=0.864, ES=0.002). Significant main effects for time were also noted for both peak (p=0.045) and mean (p=0.005) velocity (both variables decreased) during the 4-km run. No significant interactions were observed between the groups in either peak (p=0.597, ES=0.02) or mean (p=0.729, ES=0.01) velocity. The distance run at low to moderate velocities was significantly greater at Post than Pre (p=0.010) for both groups combined, however, no significant interactions were seen between BA and PL (p=0.224. ES=0.10). Similarly, a significant main effect was noted for distance run at high velocity (p=0.022). The distance run at high velocity was significantly reduced for both BA and PL, but no significant interaction was noted (p=0.363, ES=0.06). The percent distance run at low to moderate velocity was significantly increased (p=0.021) for both groups combined, but no significant interactions were observed (p=0.351, ES=0.06). The percent distance ran at high intensity was significantly lower for both groups combined (p=0.019), however, no significant interaction was observed between BA and PL (p=0.361, ES=0.06), although the decrease in both actual and percent distance run at high velocity was less in BA than in PL.

TABLE 1
Running Velocities during 4-km Run

| | | | | p | |
|---|---|---|---|---|---|
| Variable | Group | PRE | POST | Value | ES |
| Peak Velocity | BA | 5.84 ± 0.63 | 5.46 ± 0.26 | 0.597 | .02 |
| (m · sec⁻¹) | PL | 5.69 ± 0.46 | 5.51 ± 0.50 | | |
| Average Velocity | BA | 4.25 ± 0.22 | 4.13 ± 0.27 | 0.729 | .01 |
| (m · sec⁻¹) | PL | 4.18 ± 0.19 | 4.11 ± 0.19 | | |
| Low-Moderate | BA | 2811 ± 605 | 2957 ± 672 | 0.224 | .10 |
| Running Velocity | PL | 2827 ± 482 | 3297 ± 590 | | |
| (<4.4 m · sec⁻¹) | | | | | |
| High Running Velocity | BA | 1166 ± 610 | 1009 ± 675 | 0.364 | .06 |
| (>4.4 m · sec⁻¹) | PL | 1143 ± 485 | 748 ± 541 | | |
| % Distance run | BA | 70.8 ± 16.2 | 74.3 ± 18.3 | 0.351 | .06 |
| at Low to Moderate | PL | 71.3 ± 12.8 | 81.1 ± 14.4 | | |
| Running Velocity | | | | | |
| % Distance run at High | BA | 29.3 ± 16.1 | 25.4 ± 18.0 | 0.361 | .06 |
| Running Velocity | PL | 28.8 ± 13.0 | 18.9 ± 14.4 | | |
| 4K Run Time (sec) | BA | 942.4 ± 39.3 | 962.6 ± 65.0 | 0.864 | .002 |
| | PL | 949.9 ± 46.2 | 963.9 ± 44.3 | | |

ES = Effect size

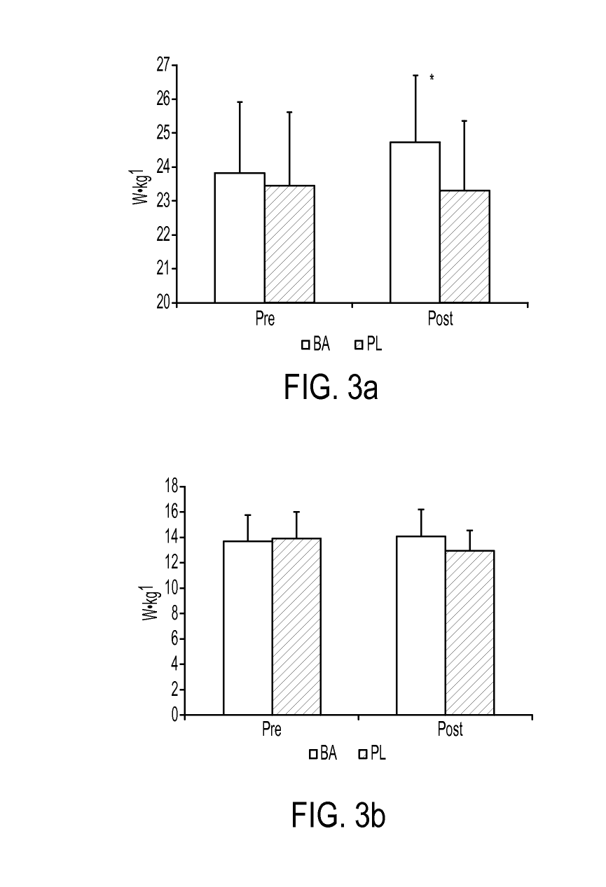

Comparisons of vertical jump relative peak and mean power performance can be observed in FIGS. 3a and 3b, respectively. Relative peak power at Post was significantly greater for BA than PL (p=0.034, ES=0.27), while relative mean power for BA at Post (14.1±1.7 w·kg-1) was 10.2% greater (p=0.139) than that observed for PL (12.8±1.5 w·kg-1). The effect size was 0.14, suggesting a large effect was due to the intervention (BA).

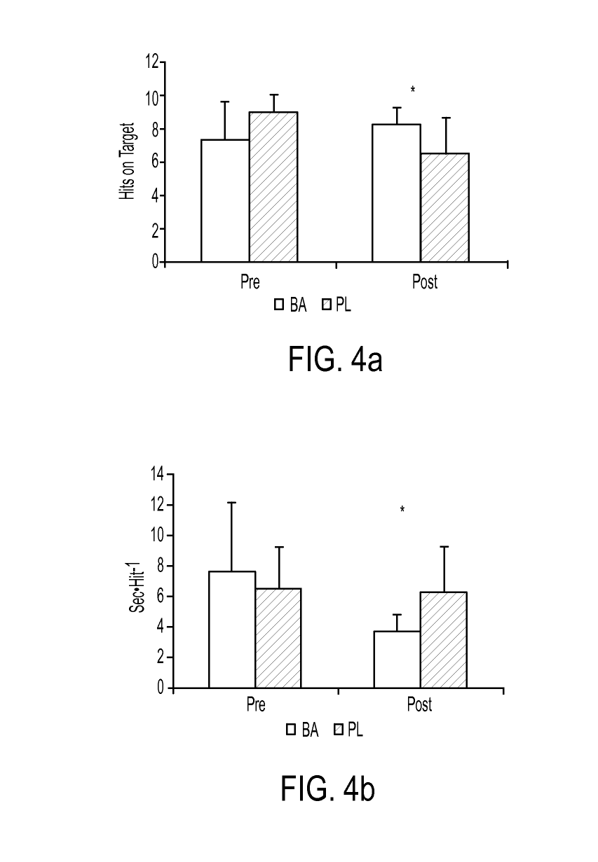

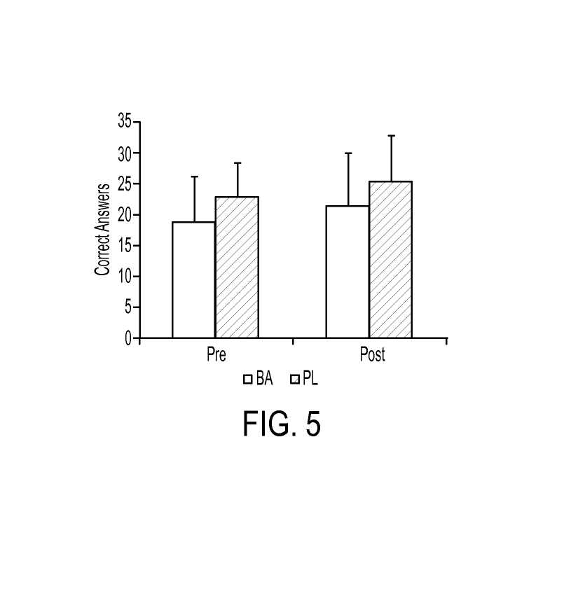

The effect of the supplement on shooting accuracy and time per shot on target can be seen in FIGS. 4a and 4b, respectively. Participants consuming BA had a significantly greater (p=0.012, ES=0.38) number of shots on target at Post (8.2±1.0) than PL (6.5±2.1). The time per shot on target at Post was also significantly greater for BA than PL (p=0.039, ES=0.27). Significant improvements from Pre to Post in the serial subtraction test was seen in both groups (p=0.014), but no significant interactions were seen between the groups (p=0.844, ES=0.003) (see FIG. 5).

During the 4-week period, all participants were in advanced military training tasks that included combat skill development, physical work under pressure, navigational training, self-defense/hand-to-hand combat and conditioning. This training program appeared to result in significant performance decrements as indicated by significant decreases in 4-km run performance in both groups. In summary, 4-weeks of beta-alanine ingestion in military personnel can enhance power performance, marksmanship and target engagement speed.

Four weeks of β-alanine ingestion with dosages similar to the one used in the present invention has been shown to elevate muscle carnosine concentrations by 60% (Hill et al., 2007). Elevations in muscle carnosine has been demonstrated to enhance intracellular muscle buffering capacity and delay fatigue during high intensity anaerobic exercise (Harris et al., 2006; Hobson et al., 2012), but it's benefits during endurance activity has proved to be inconclusive. During the 4-km run, performed in one embodiment of the invention, we were unable to show any significant advantage related to β-alanine ingestion. There have only been a limited number of studies examining the effects of β-alanine ingestion and endurance performance. Jordan and colleagues (2010) reported that following 4-weeks of β-alanine ingestion, in participants who were not training aerobically during the supplement period, a delay in blood lactate accumulation was seen, but a decrease in aerobic capacity was also noted. The physiological role of carnosine in muscle does not provide a strong mechanism for enhancing aerobic exercise performance. It may, however, increase the time spent running at higher velocities. Although our results do not support this statistically, a 34.9% difference was seen between BA and PL in the distance run at a high velocity. This was observed to have a moderate effect and warrants further exploration with larger sample sizes. Regardless, the 4-km run performed in this investigation was primarily done to increase the fatigue of the soldiers prior to the shooting and cognitive function measures.

Following the 4-km run, subjects were required to perform a jump power test. The greater power performance observed in BA compared to PL was consistent with other studies demonstrating the fatigue resistant effects of β-alanine during high intensity activity (Derave et al., 2007; Kern and Robinson, 2011; Van Thienen et al., 2009). Derave et al. (2007) reported that 4-weeks of β-alanine supplementation (4.8 g per day) was able to delay fatigue during repeated bouts of isokinetic exercise and Van Thienen and colleagues (2009) noted improved 30-sec sprint performance following a 110-min time trial. Each of those studies demonstrated a delay in fatigue following an acute exhaustive exercise protocol. Kern and Robinson (2011) reported enhanced anaerobic exercise performance following a prolonged period (8-weeks) of high intensity training in athletes supplementing with β-alanine compared to a placebo. The present invention appears to support both the acute and chronic benefits from β-alanine supplementation in delaying fatigue.

Shooting performance has been shown to be sensitive to acute fatiguing activity (Evans et al., 2003; Gillingham et al., 2004). Gillingham and colleagues (2004) demonstrated that caffeine intake before and following exhaustive exercise (2.5-hr loaded march and 1.0-hr sandbar wall construction) improve target detection, marksmanship and engagement speed during simulated combat. This present invention is the first to demonstrate that the fatigue resistant effects afforded by β-alanine ingestion can also improve marksmanship and target engagement speed following fatiguing exercise. Fatigue during sustained and highly intense combat situations may jeopardize judgment in differentiating friend from foe as quickly as possible. In this example, subjects were required to overcome a misfire in their weapon, and then complete mathematical problems while seated following their shooting performance. The participants in BA were able to perform their 10 shots (30.2±5.8 sec) faster than PL (37.7±13.9 sec), but this 24.8% difference between the groups was not statistically different (p=0.161). When the time was calculated relative to the number of shots on target, however, BA was significantly faster than PL. Furthermore, the misfire in the weapon was similar for all participants and similar in both Pre and Post assessment periods. It is possible that the familiarity with how to handle the misfire for both groups also contributed to the similar completion time for the 10 shots.

As described in the present invention, definable units, such as the military, paramilitary groups, law enforcement, medical and medical emergency units, and first responder groups such as firefighters and paramedics, now have a unique methodology for dietary supplementation across members of these units that effectively improves and maintains tactical performance and psychomotor performance. The dietary supplementation regimens demonstrated by the present invention provide these definable units with a relatively uniform issuance of safe and effective amounts of a dietary supplement, which when taken in accordance with the present invention, results in an increased ability to perform both tactical and psychomotor functions while dealing with potentially life-threatening or lifesaving situations under physically fatiguing and mentally stressful conditions.

### Study 2

### Examples

Eighteen male soldiers from an elite combat unit volunteered to participate in this double-blind study. The soldiers were not permitted to use any additional dietary supplementation and did not consume any androgens or any other performance enhancing drugs. Screening for performance enhancing drug use and additional supplementation was accomplished via a health questionnaire completed during participant recruitment. Participants were from the same unit, but were from three different squads. Volunteers from each squad were randomly assigned to one of two groups. The randomization procedure involved that each volunteer from the same squad to be alternatively assigned to each group. Using the procedures described by Gravetter and Wallnau (1996) for estimating samples sizes for repeated measures designs, a sample size of 9 of each group resulted in a statistical power (1-β) of >0.90 based on the changes in sprint performance reported by Van Thienen and colleagues (2009). The first group (BA; age 19.6±0.5 years; height: 1.76±0.05 m; body mass: 72.1±4.5 kg) consumed 6.0 g of β-alanine per day, while the second group (PL; age 20.2±1.0 years; height: 1.79±0.08; body mass: 76.4±6.1 kg) consumed 6 g of placebo (rice flour). During the 30-day study period all participants from all squads participated in the same advanced military training tasks that included combat skill development, physical work under pressure, navigational training, self-defense/hand-to-hand combat and conditioning.

Testing Protocol

This randomized, double-blind, placebo controlled investigation was conducted at the unit's training facilities, under the unit's regular training protocols and safety regulations. Data collection occurred before (PRE) and following (POST) 30 days of supplementation. During each session participants performed military relevant tasks that included a 2.5 km run, a 1-min sprint, and a 50-m casualty carry. In addition, participants performed repeated 30-m sprints in combat gear (combat vest with ammunition, helmet, and assault rifle). Between each sprint soldiers were tested on marksmanship. Immediately following the final sprint and target shooting, participants performed a 2-min serial subtraction test on the firing range to assess cognitive function under stressful conditions (continuous shooting). These assessments were based upon previously published investigations examining military performance responses during stressful conditions (Nindl, Leone et al. 2002, Harman, Gutekunst et al. 2008).

Performance Measurements

2.5 km run and 1-min Sprint

These tests simulated a rapid approach to the battlefield. Previous research has suggested that a prolonged run with sprint is a standard approach to the battlefield (Harman, Gutekunst et al. 2008). During the run and sprint all participants were dressed in shorts, T-shirt and running shoes. Both the run and sprint were performed on an asphalt road. All participants were provided with an individual global positioning system (GPS) that they wore in a vest underneath their shirt. The GPS unit (MinimaxX, V4.3, Catapult Innovations, Victoria, Australia) was positioned in a posterior pocket on the vest situated between the participant's right and left scapula in the upper-thoracic spine region. Information on velocity patterns was recorded during the 2.5 run, as well as total distance run during the 1-min sprint. During the 2.5 km run the velocity of the run was divided into three operationally distinct thresholds and defined as low speed (2.50 m˜s⁻¹-3.60 m˜s⁻¹), moderate speed (3.61 m·s⁻¹-4.43 m·s⁻¹) or high speed (>4.44 m·sec⁻¹). In addition, the average velocity and average heart rate during the 2.5 km run were also downloaded from the GPS receiver/transmitters.

During the 1-min sprint peak velocity, average velocity, total distance, total distance within 90% of peak velocity, and percent decline was downloaded from the GPS receiver/transmitters. All data were collected at 10 Hz and all analyses were performed with the system software provided by the manufacturer. The validity and reliability of the GPS technology has been previously demonstrated (Varley, Fairweather et al. 2012).

50-m Casualty Carry

This test simulated the rescue of a wounded soldier on the battlefield. This test was a modified version of that previously reported (Harman, Gutekunst et al. 2008). All participants began the test with a 60 kg manikin on their back, using a fireman's carry. On a verbal command the participant sprinted with the manikin to a cone 25-m away and returned to the starting position. All sprints were performed on a sand and dirt surface. All timing was performed with a stopwatch that measured time to the nearest 1/100th of a second. The same investigator conducted all sprint trials during PRE and POST testing.

Repeated Sprints and Shooting Performance

This test mimicked the repeated sprints and shooting engagement often accounted on a urban battlefield. The short sprints mimic the repeated rushes between points of cover during a combat situation (Harman, Gutekunst et al. 2008). Each participant began in a two point stance at the edge of the firing range in full combat gear (combat vest with ammunition, helmet, and assault rifle). Upon a verbal command the participant sprinted around a cone 15-m away and returned to the firing range. Each participant sprinted to a designated spot and lay prone on the ground as quickly as possible and delivered 3 shots to a target 30-m away. All targets were headshots and each shot that hit the target was considered accurate. Participants had 5-s to deliver three shots to the target. Upon completion of the three shots each participant pivoted and returned to the starting line and repeated the sprint and shooting sequence. A total of five sprint and shooting rounds were completed (a total of 15 shots were delivered onto the target). For safety purposes, participants did not sprint with their assault rifle. All sprints were performed on a sand and dirt surface. All timing was performed with a stopwatch that measured time to the nearest 1/100th of a second, and the same investigator conducted all sprint trials during PRE and POST testing. The number of accurate shots was recorded.

Cognitive Function

Immediately following the repeated sprints and shooting performance participants performed a modified version of the original Serial Sevens Test to analyze cognitive function (Hayman 1942). The test consisted of a two-minute timed written test in which participants were required to subtract the number 7 from a randomly generated four digit number to measure how quickly and accurately they can compute a simple mathematical problem. The four digit number appeared on the top of the first column of a three column sheet of paper. Participants were provided the sheet of paper and asked to complete as many calculations as possible in the two-minute period. The answers to the calculations were written underneath the initial number. Regardless of answer provided, participants were then required to subtract the number 7 from that new number. Participants were not told if their answer was correct or not. The number of correct answers was recorded. Intraclass correlations for this assessment has been determined in our laboratory to be R>0.81 (Wells, Hoffman et al. 2013). The test was conducted next to the firing range, and the range remained ‘hot’ (i.e., continuous shooting) throughout the two minute test.

Muscle Carnosine Content

Carnosine content of all participants was assessed by proton magnetic resonance spectroscopy (MRS) in the gastrocnemius muscle. All studies were performed on a 3-T system (Ingenia, Philips Medical Systems, Best, The Netherlands). Single-voxel, STEAM acquisitions of the gastrocnemius medialis muscle of the lower leg were carried out using a transmit-receive (16-channel) coil. In the leg the scan parameters were TR/TM/TE=2000/12/13 ms. Second-order shimming was used giving a full-width-half-maximum line width of approximately 25 Hz in the calf muscle. Water suppression was achieved by applying two bandwidth selective rf pulses. The average voxel size for the muscle spectra was 35×18×58 mm (APxRLxFH). The spectral resolution for all spectra was 0.96 Hz, and 400 averages were acquired for a scan time of 13:56 min. The spectra were analyzed using the Philips SpectroView software.

A 1-litre solution of 20 mM L-carnosine (Sigma-Aldrich) in 0.1M potassium phosphate buffer (pH=7.2) was used as an external reference phantom for absolute quantification. The following equation was used (Baguet, Bourgois et al. 2010, Derave, Everaert et al. 2010) to determine the concentration of carnosine in the gastrocnemius muscle using the C2-H peak at ˜8.0 ppm:

$$[ {C}_{m} ] = [ {C}_{r} ] \cdot \frac{{S}_{m} {V}_{r} {C}_{T 1 r} {C}_{T 2 r} {T}_{m}}{{S}_{r} {V}_{m} {C}_{T 1 m} {C}_{T 2 m} {T}_{r}}$$

Where [Cₘ], [Cᵣ] are the L-carnosine concentrations in vivo and the reference phantom, respectively; Sₘ, Sᵣ are the estimated peak areas of the C2-H carnosine peak in vivo and the reference phantom, respectively; Vₘ, Vᵣ are the volumes of the voxels in vivo and in the reference phantom, respectively; C_{T1m}, C_{T1r}, C_{T2r}, C_{T2r} are the correction factors for the T1 and T2 relaxation times in vivo and in the reference phantom, respectively; Tm, Tr are the temperatures (° K) in vivo and in the reference phantom respectively. The formulae used to calculate the correction factors were those previously recommended (Baguet, Bourgois et al. 2010, Derave, Everaert et al. 2010):

C_{T1}=[1−exp(−TR/T1)]

C_{T2}=exp(−TE/T2)

The T1, T2 values for the phantom and muscle were taken from Baguet and colleagues (2010). The correction for the coil loading was calculated according to the method previously described (Soher, van Zijl et al. 1996).

Supplement Schedule

The β-alanine supplement (CarnoSyn™) was obtained from Natural Alternatives International (San Marcos, Calif., USA). Both the supplement and placebo were in tablet form and were similar in appearance. Participants in the supplement group were provided with 2 tablets of sustained-release β-alanine at a dose of (2 g per serving) three times per day (total β-alanine intake was 6 g per day) and subjects in the placebo group were provided with an equivalent amount of rice powder. Participants were instructed to consume the supplement following their meals with water. Each participant was provided with a bottle containing a week's supply of tablets. All bottles were returned at the end of the week. All tablets left in the bottle were counted, recorded, and the next week's bottle was provided to the participant. Supplementation occurred every day over a 30-day period.

Statistical Analysis

Data was analyzed using a 2×2 [treatment (BA, PL) X time (PRE, POST)] repeated measures analysis of variance. In the event of a significant F ratio, LSD post-hoc comparisons were used. POST-PRE (A) performance changes were analyzed using an unpaired t test. Due to logistical issues only 10 of the 18 participants were able to have their muscle carnosine concentrations assessed at the PRE assessment. As such, comparisons A carnosine content was analyzed using the non-parametric independent samples median test. An alpha level of p≦0.05 was considered statistically significant for all comparisons. Results were considered significant at an alpha level of p≦0.05. Spearman rank correlation analysis was used to examine the relationship between changes in carnosine content and performance measures. All data are reported as mean±SD. Data were analyzed using SPSS v20 software (SPSS Inc., Chicago, Ill.).

Results

Compliance for consuming the supplement or placebo was 100%. No adverse events were reported from participants in either group during the duration of the study. Body mass of the participants did not change (p=0.50) from PRE (74.2±5.7 kg) to POST (74.1±5.8 kg) assessments, and no differences were noted in comparisons between groups.

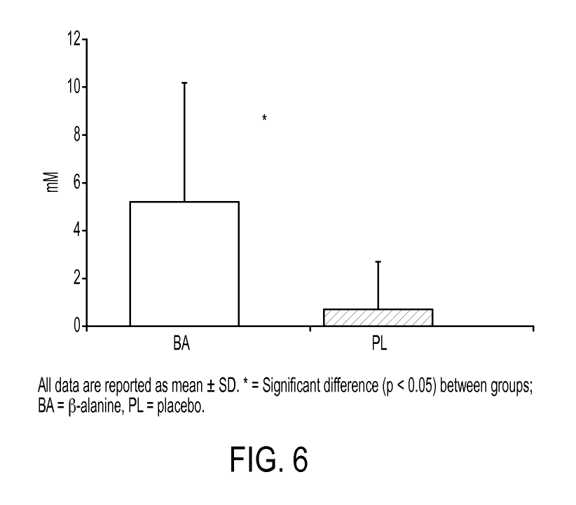

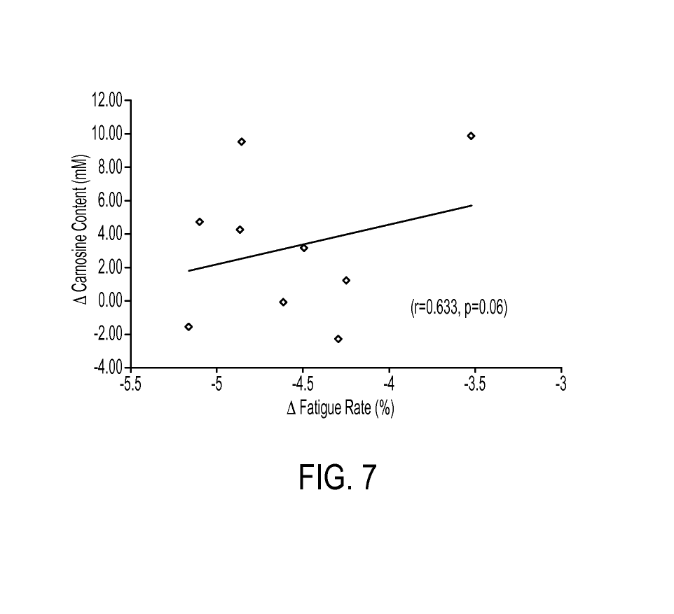

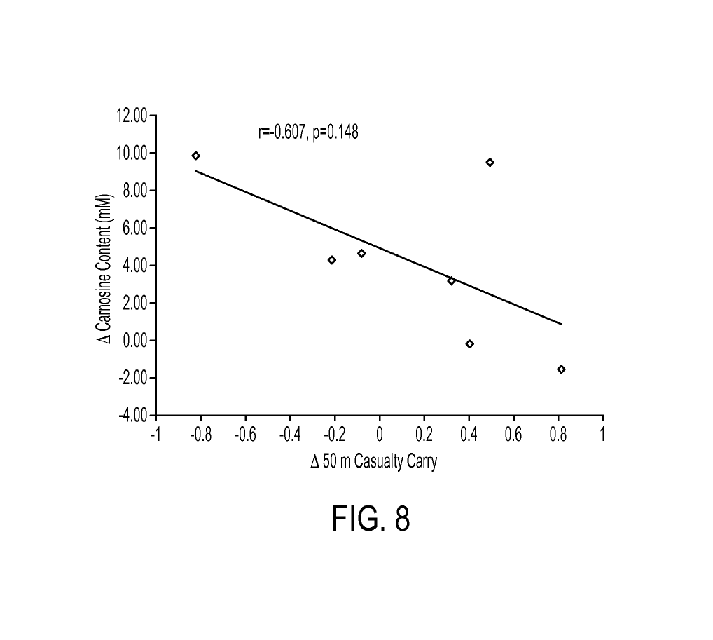

Comparisons in the Δ carnosine content within the gastrocnemius muscle are shown in FIG. 6. Baseline carnosine content (6.7±2.2 mM) in both groups was similar to that previously published (Baguet, Bourgois et al. 2010, Stellingwerff, Anwander et al. 2012). Significant elevations (p=0.048) from baseline was noted in BA compared to PL. Changes in the carnosine content of the gastrocnemius were moderately correlated to changes in fatigue rate in the 1-min sprint (r=0.633, p=0.06) (FIG. 7) and in the 50-m casualty carry (r=−0.607, p=0.148) (FIG. 8). Although these correlations were not statistically different they did indicate a trend towards a relationship between the change in muscle carnosine content and performance.

Comparisons between BA and PL in the measures examined during the 2.5 km run are described in Table 2. No significant differences were noted between the groups in the time for the 2.5 km run (p=0.866), average velocity (p=0.944) and average heart rate (p=0.122). In addition, no significant differences were noted between the groups in the percent of distance run at low, (p=0.873), medium (p=0.502) and high intensity (p=0.605). A moderate, but non-significant correlation (r=0.538, p=0.135) was seen between the change in muscle carnosine content and the change in distance run at a moderate intensity.

TABLE 2
Performance Variables during the 2.5 km Run

| Variable | Group | PRE | POST | p Value |
|---|---|---|---|---|
| Time | BA | 624 ± 22.6 | 629 ± 23.8 | 0.866 |
| (sec) | PL | 633 ± 25.3 | 609 ± 36.4 | |
| Average Velocity | BA | 3.97 ± 0.17 | 4.09 ± 0.25 | 0.944 |
| (m · s⁻¹) | PL | 3.94 ± 0.14 | 4.05 ± 0.24 | |
| Average Heart Rate | BA | 156.8 ± 15.5 | 165.7 ± 6.1 | 0.122 |
| (beats · min⁻¹) | PL | 162.1 ± 11.7 | 159.3 ± 11.4 | |
| Distance Run at Low | BA | 12.3 ± 12.0 | 10.7 ± 14.0 | 0.873 |
| Intensity (%) | PL | 15.2 ± 12.6 | 12.3 ± 14.9 | |
| Distance Run at | BA | 69.8 ± 12.1 | 65.1 ± 14.8 | 0.502 |
| Moderate Intensity | PL | 71.1 ± 10.9 | 63.7 ± 14.7 | |
| (%) | | | | |
| Distance Run at High | BA | 18.4 ± 12.4 | 23.9 ± 20.1 | 0.645 |
| Intensity (%) | PL | 13.3 ± 9.2 | 23.9 ± 20.2 | |

All data are reported as mean ± SD

During the 1-min sprint no significant difference (p=0.723) was observed in the total distance run from PRE (310.0±16.7 m vs. 310.7±23.7 m) to POST (302.4±21.2 m vs. 306.6±17.2 m) in either BA or PL, respectively, and no between group differences were noted as well. Similarly, no significant changes in either group were noted in peak or mean velocity, fatigue rate and the distance run at 90% of peak velocity (see Table 3). In addition, no between group differences were noted in any of the measured variables.

TABLE 3
Performance Variables during the Sprint Protocols

| Assessment | Variable | Group | PRE | POST | p Value |
|---|---|---|---|---|---|
| 1-min Sprint | Peak Velocity | BA | 6.68 ± 0.36 | 6.57 ± 0.18 | 0.354 |
| | (m · s⁻¹) | PL | 6.71 ± 0.48 | 6.77 ± 0.43 | |
| | Average Velocity | BA | 5.21 ± 0.28 | 5.09 ± 0.36 | 0.535 |
| | (m · s⁻¹) | PL | 5.19 ± 0.33 | 5.15 ± 0.29 | |
| | Fatigue rate | BA | 32.5 ± 5.5 | 30.9 ± 6.7 | 0.893 |
| | (%) | PL | 32.2 ± 7.1 | 30.1 ± 5.4 | |
| | Distance Run at | BA | 69.5 ± 4.1 | 58.5 ± 7.7 | 0.199 |
| | 90% Peak Velocity | PL | 66.0 ± 6.0 | 60.8 ± 7.5 | |
| Repeat 30-m | Average Sprint | BA | 7.42 ± 0.24 | 8.00 ± 0.29 | 0.780 |
| Sprints | Time (s) | PL | 7.43 ± 0.26 | 8.00 ± 0.22 | |
| | Fatigue rate (%) | BA | 91.6 ± 4.8 | 91.8 ± 3.0 | 0.432 |
| | | PL | 93.8 ± 1.8 | 92.4 ± 3.2 | |

All data are reported as mean ± SD

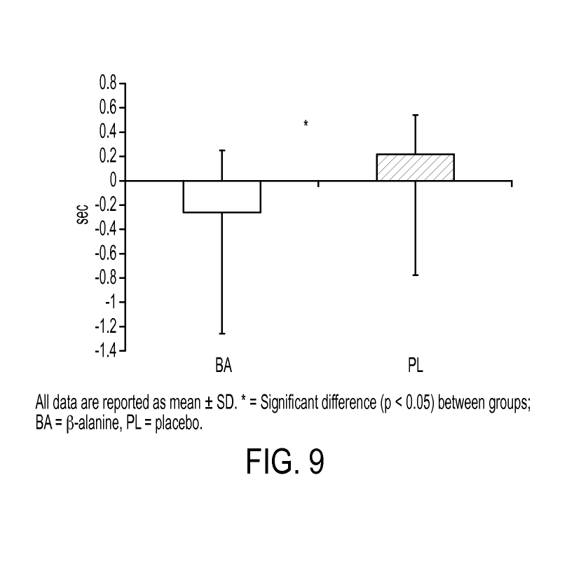

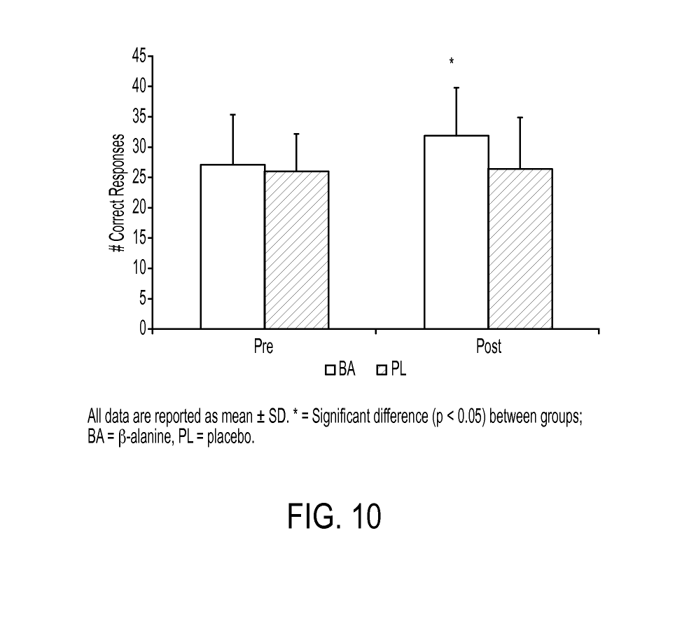

Changes in the Δ time for the 50-m casualty carry are depicted in FIG. 9. Participants in BA significantly (p=0.044) improved their time for the sprint compared to PL. A significant difference (p=0.022) was observed in the serial subtraction test under stress (see FIG. 10). Ingestion of β-alanine for 30-days appeared to significantly improve performance compared to placebo.

Discussion

Results of this study demonstrate that 30-days of β-alanine ingestion was effective in elevating muscle carnosine content in the gastrocnemius muscle of elite combat soldiers during a period of high intensity training. In addition, the increase in β-alanine ingestion appeared to enhance measures of high intensity military performance, and improve cognitive function. These results confirm the results of Study 1, above, that there is a benefit of (3-alanine ingestion on military personnel, and are consistent regarding elevations in muscle carnosine content. For example, this study demonstrated significant performance improvements in the 50-m casualty carry.

The 50-m casualty carry example did result in significant performance improvements for BA compared to PL. Following the 30-day supplement period, participants consuming β-alanine performed the sprint faster than those participants consuming the placebo. Although the duration of the sprint ranged from 13.72 s to 17.18 s the added resistance provided by sprinting with a manikin in dirt and sand added a significant stress to the anaerobic energy system. The 60 kg manikin was approximately 81% of the body mass of the average participant. A load of this magnitude has been shown to significantly enhance the metabolic cost associated with activity (Knapik, Reynolds et al. 2004), and is a stress commonly reported among infantry soldiers who carry between 29 kg-60 kg in their backpacks during various military specific tasks (Nindl, Castellani et al. 2013).

This study also reported significant improvements in cognitive performance, as assessed by the 2-min serial subtraction test. The participants in this present study were required to maintain their focus despite the active firing line that was occurring near them. The loud noise of the firing range coupled with the stress of performing mathematical problems may have contributed to a high level of anxiety within the participants. A recent study has indicated that anxiety can significantly decrease cognitive performance, and specifically mathematical skills in infantry soldiers (Nibbeling, Oudejans et al. 2014). The results of this present study indicate that 30-days of β-alanine ingestion enhances cognitive function to a greater extent than a placebo.

In conclusion, the results of this study indicate that 30-days of β-alanine ingestion in soldiers of an elite combat unit can increase muscle carnosine content and improve military specific performance. Further, changes in muscle carnosine content were moderately correlated to changes in fatigue rate during prolonged sprint activity. Also, cognitive performance under stressful conditions were significantly greater in participants consuming β-alanine compared to placebo.

Although the foregoing description is directed to the certain embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

1. A method of maintaining or improving tactical performance and/or psychomotor performance, the method comprising:
administering a human dietary supplement comprising a free amino acid beta-alanine, or a salt thereof, over a period of time in an effective amount to maintain or improve tactical performance and/or psychomotor performance.

2. The method of claim 1, wherein the individual is performing fatiguing, high intensity physical activity and/or is functioning in a stressful environment.

3. The method of claim 1, wherein the human dietary supplement is administered one or more times per day for multiple days.

4. The method of claim 3, wherein the effective amount is at least about 3.0 g of the free amino acid beta-alanine, or salt thereof.

5-6. (canceled)

7. The method of claim 3, wherein the multiple days allows for non-supplementation days.

8. The method of claim 7, wherein the non-supplementation is no more than one consecutive day and no more than two days in a seven day period.

9. The method of claim 1, wherein the human dietary supplement may be provided with a form of creatine, an insulin stimulator, a carbohydrate, vitamins, other proteins and amino acids, or combinations thereof.

10. (canceled)

11. The method of claim 1, wherein the administering step is prior to the tactical performance and/or psychomotor performance, during the tactical performance and/or psychomotor performance, or after the tactical performance and/or psychomotor performance.

12-20. (canceled)

21. A method of improving target acquisition, accuracy and engagement times by an individual, said method comprising:
administering a human dietary supplement comprising a free amino acid beta-alanine, or a salt thereof, over a period of time in an effective amount to improve the target acquisition, accuracy and engagement times by the individual.

22. The method of claim 21, wherein the human dietary supplement is administered one or more times per day for multiple days.

23. The method of claim 22, wherein the multiple days allows for non-supplementation days.

24. The method of claim 22, wherein the human dietary supplement may be provided with a form of creatine, an insulin stimulator, a carbohydrate, vitamins, other proteins and amino acids, or combinations thereof.

25. The method of claim 21, comprising the step of adjusting the effective amount once the improved target acquisition is achieved to allow for a maintenance phase.

26. A method comprising administering a human dietary supplement comprising a free amino acid beta-alanine, or a salt thereof, over a period of time in an effective amount,
wherein the method:
increases military readiness;
improves shooting accuracy after physical exertion;
decreases reaction time;
decreases involuntary muscle action or movement caused by physical stress during critical actions;
increases control of breathing or breath during and after physically fatiguing and stressful situations; or
combinations thereof.

27. The method of claim 26, wherein the human dietary supplement is administered one or more times per day for multiple days.

28. The method of claim 27, wherein the effective amount is at least about 3.0 g of the free amino acid beta-alaine, or salt thereof.

29. The method of claim 26, wherein the human dietary supplement is not from a natural or conventional food.

30. (canceled)

31. The method of claim 27, wherein the multiple days allows for non-supplementation days.

32. The method of claim 31, wherein the non-supplementation is no more than one consecutive day and no more than two days in a seven day period.

33. The method of claim 26, wherein the human dietary supplement may be provided with a form of creatine, an insulin stimulator, a carbohydrate, vitamins, other proteins and amino acids, or combinations thereof.

34-35. (canceled)
